SEQUENCE LISTING (8 sequences)

SEQ ID NO: 1 - protein, 9 aa, Artificial
  Synthetic peptide containing BACE1 cleavage site of human wild-type APP
GGGISEVKM

SEQ ID NO: 2 - protein, 9 aa, Artificial
  Synthetic peptide containing BACE1 cleavage site of human Swedish FAD variant of APP
GGGISEVNL

SEQ ID NO: 3 - DNA, 33 bp, Artificial
  PCR Primer
tagtagcgaggatccagcttcagcagggagatg

SEQ ID NO: 4 - DNA, 28 bp, Artificial
  Primer
tcattcaagcttatggcccaagccctgc

SEQ ID NO: 5 - DNA, 36 bp, Artificial
  Primer
tagcgatctagattacttgtacagctcgtccatgcc

SEQ ID NO: 6 - DNA, 32 bp, Artificial
  Primer
tcattcgcggccgcctagctagagatccctcg

SEQ ID NO: 7 - DNA, 30 bp, Artificial
  Primer
tagcgaggtaccggccgcttagttctgcat

SEQ ID NO: 8 - protein, 23 aa, Artificial
  C-terminal peptide of human APP
HLSKMQQNGYENPTYKFFEQMQN

(19) United States
(12) Patent Application Publication
(10) Pub. No.: US 2010/0221760 A1
(43) Pub. Date: Sep. 2, 2010

(54) METHODS AND COMPOSITIONS FOR DETECTING AND QUANTIFYING sAPPbeta

(76) Inventors: TAE-WAN KIM, EAST BRUNSWICK, NJ (US); JEREMY C. HWANG, GREAT NECK, NY (US)

Correspondence Address: BAKER BOTTS L.L.P., 30 ROCKEFELLER PLAZA, 44TH FLOOR, NEW YORK, NY, 10112-4498, US

(21) Appl. No.: 12/710,965

(22) Filed: Feb. 23, 2010

Related U.S. Application Data
(62) Division of application No. 12/353,162, filed on Jan. 13, 2009, now abandoned.
Application No. 12/353,162 is a continuation of application No. PCT/US07/15938, filed on Jul. 13, 2007.
(60) Provisional application No. 60/830,998, filed on Jul. 14, 2006.

(51) Int. Cl. G01N 33/53

(52) U.S. Cl. 435/7.94

(57) ABSTRACT

The present invention provides methods (assays) for detecting and/or quantifying sAPPβ, a secreted β-secretase (BACE1) cleavage fragment of the β-amyloid precursor protein (APP), in a biological sample. One such method includes contacting a biological sample with a first antibody that selectively binds to a BACE1 cleavage site on sAPPβ and detecting the presence of the antibody. Also provided are compositions, including antibodies that selectively bind to the BACE1 cleavage site of sAPPβ. Kits containing such compositions are also provided. Methods of diagnosing a neurodegenerative disease, such as AD, using the methods and compositions of the present invention are further provided. Methods for identifying BACE1 modulators, candidate compounds that are BACE1 modulators, and methods for treating, preventing or ameliorating neurodegenerative disease, such as AD, using such compounds or pharmaceutical compositions containing such compounds are also provided.

### CROSS-REFERENCE TO RELATED APPLICATIONS

This application claims the benefit of U.S. Provisional Application Ser. No. 60/830,998, filed Jul. 14, 2006. The entire disclosure of this application is relied upon and incorporated by reference herein.

### GOVERNMENT FUNDING

Work described herein was funded, in whole or in part, by National institutes of Health Training Grant 5T32 GM07367-31. The United States government may have certain rights in the invention.

### FIELD OF THE INVENTION

The present invention relates to methods and compositions for detecting and/or quantifying sAPPβ, a secreted β-secretase (BACE1) cleavage fragment of the β-amyloid precursor protein (APP). More particularly, the present invention relates to compositions, including antibodies that selectively bind to the BACE1 cleavage site of sAPPβ, transfected cells that express BACE1, APP, and reporter genes, and vectors that encode such polypeptides. The present invention further relates to the use of such compositions in various methods (assays) for detecting and/or quantitating BACE1. The present invention also relates to methods for diagnosing a neurodegenerative disorder, such as Alzheimer's Disease (AD) using such compositions. The present invention further relates to methods for identifying BACE1 modulators, candidate compounds that are BACE1 modulators, and methods for treating, preventing, or ameliorating neurodegenerative diseases, such as AD, using such candidate compounds or pharmaceutical compositions containing such candidate compounds.

### BACKGROUND OF THE INVENTION

### Alzheimer's Disease and Pathogenesis

Alzheimer's Disease is a progressive neurodegenerative disease characterized by progressive memory deficits, impaired cognitive function, altered and inappropriate behavior, and a progressive decline in language function. It is the most prevalent age-related dementia, affecting an estimated 18 million people worldwide, according to the World Health Organization. As medical advances continue to prolong the human lifespan, it is certain that AD will affect an increasing proportion of the population. There is no cure for AD, and current FDA-approved therapies provide only temporary and symptomatic relief, while doing little to counteract disease progression.

Pathologically, AD patients display cortical atrophy, loss of neurons and synapses, and hallmark extracellular senile plaques and intracellular neurofibrillary tangles. Senile (or neuritic) plaques are composed of aggregated amyloid β-peptide (Aβ), and are found in large numbers in the limbic and association cortices (1). It is widely hypothesized that the extracellular accumulation of Aβ contributes to axonal and dendritic injury and subsequent neuronal death. Neurofibrillary tangles consist of pairs of ˜10 nm filaments wound into helices (paired helical filaments or PHF). Immunohistochemical and biochemical analysis of neurofibrillary tangles revealed that they are composed of a hyperphosphorylated form of the microtubule-associated protein tau. These two classical pathological lesions of AD can occur independently of each other. However, there is growing evidence that the gradual accumulation of Aβ and Aβ-associated molecules leads to the formation of neurofibrillary tangles (1). As such, much research is directed at inhibiting the generation of the amyloid β-peptide.

### Secretase-Mediated Processing of APP

Aβ is derived from the sequential cleavage of APP by membrane-bound proteases known as β-secretase and γ-secretase. A competing proteolytic pathway to the β-secretase pathway exists—the α-secretase pathway—which results in cleavage of APP within the Aβ domain, thereby precluding the generation of Aβ.

β-site APP cleavage enzyme 1 (BACE1) was identified as the major β-secretase activity that mediates the first cleavage of APP in the β-amyloidgenic pathway (2-5). BACE1 is a 501 amino acid protein that bears homology to eukaryotic aspartic proteases, especially from the pepsin family (6). In common with other aspartic proteases, BACE1 is synthesized as a zymogen with a pro-domain that is cleaved by furin to release the mature protein. BACE1 is a type I transmembrane protein with a lumenal active site that cleaves APP to release an ectodomain (sAPPβ) into the extracellular space. The remaining C-terminal fragment (CTF) undergoes subsequent cleavage by γ-secretase to release Aβ and the APP intracellular C-terminal domain (AICD). The presenilins have been proposed to be the major enzymatic component of γ-secretase, whose imprecise cleavage of APP produces a spectrum of Aβ peptides varying in length by a few amino acids at the C-terminus. The majority of Aβ normally ends at amino acid 40 (Aβ₄₀), but the 42-amino acid variant (Aβ₄₂) has been shown to be more susceptible to aggregation, and has been hypothesized to nucleate senile plaque formation.

The competing α-secretase pathway is the result of sequential cleavages by α- and γ-secretase. Three metalloproteases of the A Disintegrin And Metalloprotease family (ADAM 9, 10, and 17) have been proposed as candidates for the α-secretase activity, which cleaves APP at position 16 within the Aβ sequence (7-9). This cleavage also releases an ectodomain (sAPPα), which seems to have neuroprotective functions (10). Subsequent cleavage of the 83-amino acid CTF (C83) releases p3, which is non-amyloidgenic, and the AICD. The functions of these fragments are not known, although AICD is hypothesized to mediate intracellular signaling, based on analogy to the intracellular C-terminal domain of Notch.

### BACE1 Inhibitors and Therapeutic Interventions in AD

BACE1 has become a popular research topic since its discovery, and has, perhaps, surpassed γ-secretase as the most promising target for pharmaceutical research. γ-Secretase is known to cleave Notch, which serves important functions in neuronal development. Presenilin knockout mice demonstrated abnormal somitogenesis and axial skeletal development with shortened body length, as well as cerebral hemorrhages (11, 12). In contrast, several groups reported that BACE1 knockout mice are healthy and show no signs of adverse effect (13, 14), while one group noticed subtle neurochemical deficits and behavioral changes in otherwise viable and fertile mice (15). Although recent studies have shown that BACE1 knockout mice exhibit hypomyelination of peripheral nerves (16), the consequences of BACE1 inhibition in adult animals—where myelination has already taken place—is unclear. Thus, BACE1 remains a hopeful candidate for AD therapeutics research.

Molecular modeling (17) and subsequent X-ray crystallography (18) of the BACE1 active site complexed with a transition-state inhibitor provided crucial information about BACE1-substrate interactions. Structurally, the BACE1 active site is more open and less hydrophobic than that of other aspartic proteases. Peptide inhibitors of BACE1 such as P4′StatVal and OM99-2 have been developed to explore the structure and kinetics of BACE1.

Small molecule BACE1 inhibitors are also being developed by numerous investigators. In particular, Hussain et. al. have demonstrated in vivo efficacy of their BACE1 small molecule inhibitor, GSK188909, in a mouse model of AD (19). While these results are promising, many challenges still remain.

Because BACE1 has a large active site, it is difficult to design a compound large enough to achieve the high specificity required for a drug, yet be small enough to effectively traverse the blood-brain barrier. In fact, because of low brain penetration, a p-glycoprotein inhibitor was required to increase transport of GSK188909 across the blood-brain barrier (19). Furthermore, BACE1 has been reported to cleave multiple substrates, including ST6Gal I, PSGL-1, β subunits of voltage-gated sodium channels, APP-like proteins (APLPs), LDL receptor related protein (LRP), Aβ, and, most recently, type III neuregulin 1 (NRG1). The consequences of inhibiting BACE1 directly are therefore not clearly understood. Accordingly, it would be desirable to have a novel cell-based assay to monitor BACE1-mediated cleavage of APP. In such a method, because a cleavage product (sAPPβ) is measured, the assay may be used to identify a diverse set of small molecules with a variety of mechanisms of action.

Moreover, although commercial ELISA kits are available for detecting various forms of Aβ, none currently exist that detect sAPPβ, the species that most directly correlates with BACE1 activity. Considering the drawbacks noted above and the importance of BACE1 in AD, it would be advantageous to be able to detect sAPPβ based on cleavage site-specific antibodies for sAPPβ that discriminate against sAPPα. The present invention is directed to meeting these and other needs.

### SUMMARY OF THE INVENTION

One embodiment of the present invention is a method for detecting the presence of sAPPβ, a secreted β-secretase (BACE1) cleavage fragment of the beta-amyloid precursor protein (APP) in a biological sample. This method comprises (a) contacting a biological sample with a first antibody that selectively binds to a BACE1 cleavage site on sAPPβ and (b) detecting the presence of the antibody.

Another embodiment of the present invention is a method for diagnosing a neurodegenerative disorder in a subject. This method comprises (a) providing a first antibody that selectively binds to sAPPβ, a secreted β-secretase (BACE1) cleavage fragment of the beta-amyloid precursor protein (APP), but not to sAPPα, a secreted α-secretase cleavage fragment of the APP, (b) contacting a sample from a subject with the first antibody; (c) detecting the presence of the first antibody selectively bound to the sAPPβ; and (d) correlating the presence of the first antibody in step (c) with a neurodegenerative disorder or a predisposition to develop the neurodegenerative disorder.

Another embodiment of the present invention is a method for detecting and/or quantifying β-secretase (BACE1) activity in a biological sample. This method comprises (a) contacting a biological sample with a first antibody that selectively binds to sAPPβ, a secreted BACE1 cleavage fragment of the beta-amyloid precursor protein (APP), but not to sAPPα, a secreted α-secretase cleavage fragment of APP, (b) detecting and/or quantifying the amount, if any, of sAPPβ in the biological sample; and (c) correlating the quantity of sAPPβ in the sample with BACE1 activity.

Another embodiment of the present invention is a method for identifying a compound that modulates β-secretase (BACE1) activity. This method comprises (a) providing, in a suitable media, a cell line transfected with a construct comprising a polynucleotide encoding BACE1 and a polynucleotide encoding a β-amyloid precursor protein (APP); (b) contacting the cell line with a candidate compound, and (c) determining whether the candidate compound modulates BACE1 activity, wherein a change in the level of sAPPβ, a secreted BACE1 cleavage fragment of APP, compared to a control cell line that was not contacted with the candidate compound, indicates that the candidate compound modulates the activity of BACE1.

Another embodiment of the present invention is a method for identifying a compound that modulates β-secretase (BACE1) activity. This method comprises (a) contacting a biological sample comprising sAPPβ, a secreted BACE1 cleavage fragment of the beta-amyloid precursor protein (APP) with a solid support comprising a surface to which is immobilized a first antibody that selectively binds to the N-terminal portion of sAPPβ, wherein the first antibody captures the sAPPβ in the sample, (b) contacting the solid support with a solution comprising a second antibody that selectively binds to a BACE1 cleavage site on the captured sAPPβ, and (c) detecting the presence of the captured sAPPβ on the solid support. This embodiment further includes determining whether a candidate compound administered to a patient prior to withdrawal of the biological sample modulated BACE1 levels compared to a biological sample withdrawn from the patient prior to administration of the candidate compound.

Another embodiment of the invention is a method for identifying a candidate compound that modulates β-secretase (BACE1) activity. This method comprises (a) contacting a biological sample comprising sAPPβ, a secreted BACE1 cleavage fragment of the beta-amyloid precursor protein (APP) with a solid support comprising a surface to which is immobilized a first antibody that selectively binds to a BACE1 cleavage site on the sAPPβ, wherein the first antibody captures the sAPPβ in the sample, (b) contacting the solid support with a solution comprising a second antibody that selectively binds to the N-terminal portion of the captured sAPPβ, and (c) detecting the presence of the captured sAPPβ on the solid support. This embodiment further includes determining whether a candidate compound administered to a patient prior to withdrawal of the biological sample modulated BACE1 levels compared to a biological sample withdrawn from the patient prior to administration of the candidate compound.

Another embodiment of the invention is a method of identifying a compound that modulates β-secretase (BACE1) activity. This method comprises (a) providing cells in an appropriate media, which cells are transfected with a construct comprising a polynucleotide encoding BACE1, a polynucleotide encoding a first reporter gene, a polynucleotide encoding a β-amyloid precursor protein (APP), and a polynucleotide encoding a second reporter gene, (b) contacting the cells with a candidate compound, (c) contacting a sample of the cell media in (b) with a solid support having immobilized on a surface thereof an antibody that selectively binds to a BACE1 cleavage site on sAPPβ, a secreted BACE1 cleavage fragment of the APP, (d) detecting the presence of the second reporter gene product in the media, and (e) correlating the relative quantity of the second reporter gene product in the media with an ability of the candidate compound to modulate BACE1 activity.

Another embodiment of the invention is a kit comprising, packaged together, a vial containing a lyophilized first antibody that selectively binds to a β-secretase (BACE1) cleavage site on sAPPβ, a secreted BACE1 cleavage fragment of the beta-amyloid precursor protein (APP) and a vial containing a lyophilized second antibody that selectively binds to the N-terminal portion of sAPPβ.

Another embodiment of the invention is a kit comprising, packaged together, a vial containing a lyophilized antibody that selectively binds to a β-secretase (BACE1) cleavage site on sAPPβ, a secreted BACE1 cleavage fragment of the beta-amyloid precursor protein (APP) and a vial containing a lyophilized vector comprising a first construct comprising a polynucleotide encoding BACE1 and a first reporter gene and a second construct comprising a polynucleotide encoding the APP and a second reporter gene.

Another embodiment of the invention is an antibody that selectively binds to a β-secretase (BACE1) cleavage site on a sAPPβ, a secreted BACE1 cleavage fragment of the beta-amyloid precursor protein (APP).

Another embodiment of the present invention is a method for identifying a molecule that modulates β-secretase (BACE1) activity. This method comprises (a) providing culture media from a cell culture contacted with or transfected with a molecule, wherein the cells from the cell culture, prior to transfection, shed sAPPβ into the media, (b) contacting the media from (a) with a solid support comprising a surface to which is immobilized a first antibody that selectively binds to the N-terminal portion of sAPPβ, wherein the first antibody captures the sAPPβ in the sample, (c) contacting the solid support with a solution comprising a second antibody that selectively binds to a BACE1 cleavage site on the captured sAPPβ, and (d) detecting the presence of the captured sAPPβ on the solid support, wherein a change in the level of sAPPβ detected in the contacted or transfected cells compared to control cells that were not contacted or transfected with the molecule is an indication that the molecule modulates BACE1 activity.

Another embodiment of the present invention is a method for identifying a molecule that modulates β-secretase (BACE1) activity. This method comprises (a) providing culture media from a cell culture contacted with or transfected with a molecule, wherein the cells from the cell culture, prior to transfection, shed sAPPβ into the media, (b) contacting the culture media from (a) with a solid support comprising a surface to which is immobilized a first antibody that selectively binds to a BACE1 cleavage site on the sAPPβ, wherein the first antibody captures the sAPPβ in the sample, (c) contacting the solid support with a solution comprising a second antibody that selectively binds to the N-terminal portion of the captured sAPPβ, and (d) detecting the presence of the captured sAPPβ on the solid support, wherein a change in the level of sAPPβ detected in the contacted or transfected cells compared to control cells that were not contacted or transfected with the molecule is an indication that the molecule modulates BACE1 activity.

A further embodiment of the present invention is a high-throughput screening method for identifying a molecule that modulates β-secretase (BACE1) activity. This method comprises (a) providing cells in an appropriate media, which cells are transfected with a construct comprising a polynucleotide encoding BACE1, a polynucleotide encoding a first reporter gene, a polynucleotide encoding a β-amyloid precursor protein (APP), and a polynucleotide encoding a second reporter gene, (b) introducing a test molecule into the cells, (c) contacting a sample of the cell media in (b) with a solid support having immobilized on a surface thereof an antibody that selectively binds to a BACE1 cleavage site on sAPPβ, a secreted BACE1 cleavage fragment of APP, (d) detecting the presence of the second reporter gene product in the media, and (e) correlating the relative quantity of the second reporter gene product in the media with an ability of the test molecule to modulate BACE1 activity.

Another embodiment of the invention is a method of identifying a compound that modulates β-secretase (BACE1) activity. This method comprises (a) providing cells in an appropriate media, which cells are transfected with pBudCE4.1/BACEGFP-SEAPAPPwt, (b) contacting the cells with a candidate compound, (c) contacting a sample of the cell media in (b) with a solid support having immobilized on a surface thereof an antibody that selectively binds to a BACE1 cleavage site on sAPPβ, a secreted BACE1 cleavage fragment of APP, (d) detecting the presence of the secreted alkaline phosphatase (SEAP) gene product in the media; and (e) correlating the relative quantity of the SEAP gene product in the media with an ability of the candidate compound to modulate BACE1 activity.

Another embodiment of the invention is a compound identified according to any of the screening methods of the present invention. For example, the compound may be Compound 1, Compound 2, or Compound 3. This embodiment also, where appropriate, embraces analogs, enantiomers, optical isomers, diastereomers, N-oxides, crystalline forms, hydrates, and pharmaceutically acceptable salts of the compounds of the present invention, and combinations thereof.

Another embodiment of the present invention is a pharmaceutical composition comprising at least one compound of the present invention or analogs, enantiomers, optical isomers, diastereomers, N-oxides, crystalline forms, hydrates, and pharmaceutically acceptable salts of the compounds of the present invention, and/or combinations thereof.

A further embodiment of the invention is a method of treating, preventing, or ameliorating the effects of a subject suffering from a neurodegenerative disorder. This method comprises administering to a subject in need thereof an amount of at least one compound or composition of the present invention that is effective to modulate BACE1 levels in the subject.

### BRIEF DESCRIPTION OF THE DRAWINGS

FIG. 1 is a schematic that shows β-site cleavage-specific antibodies of the present invention against the wild-type or Swedish mutant form of human β-amyloid precursor protein (APP).

FIG. 2 is a schematic illustration of certain of the sAPPβ detection assays according to the present invention.

FIG. 3 shows a Western blot analysis of nine G418-selected Neuro2a-BACE stable cell colonies. Equal protein concentrations of lysate were loaded. BACE-myc was visualized with the anti-myc antibody 9E10.

FIGS. 4A-C demonstrate the specificity of the sAPPβ antibodies according to the present invention. FIG. 4C further shows the characterization of pBudCE4.1/BACEGFP-SEAPAPP constructs.

FIG. 5 shows a vector map for pBudCE4.1/BACEGFP-SEAPAPP.

FIGS. 6A and B show data from a sAPPβ sandwich ELISA according to the present invention employing an APP N-terminal capture antibody and a sAPPβ-specific detection antibody (Method 1A).

FIGS. 7A and B show data from a sAPPβ sandwich ELISA according to the present invention employing a sAPPβ-specific capture antibody and an APP N-terminal detection antibody (Method 1B).

FIGS. 8A-F show various data from a Hybrid ELISA-SEAP assay according to the present invention for sAPPβ detection (Method 2).

FIG. 9 shows the characterization of SY5Y-BACEGFP-SEAPAPPwt stable cells.

FIG. 10 is a schematic showing a cell-based BACE1 screening assay according to the present invention.

FIG. 11 shows a validation for a BACE1 assay according to the present invention, including Z-values.

FIG. 12 is a bar graph showing the results of a BACE1 assay using SY5Y-BACEGFP-SEAPAPPwt stable cells.

FIG. 13 is a graph showing a BACE Inhibitor IV dose-response curve using SY5Y-BACEGFP-SEAPAPPwt stable cells.

FIG. 14 is a graph summarizing a primary screening of about 3000 compounds.

FIG. 15A is a table summarizing the results of a rescreen for certain selected candidate compounds. FIG. 15B shows the structure of the three best hit compounds.

### DETAILED DESCRIPTION OF THE INVENTION

The present invention is directed towards methods and compositions for detecting and/or quantifying sAPPβ and to the use of such compositions in various methods (assays) for detecting and/or quantitating BACE1. The present invention is also directed to methods for diagnosing a neurodegenerative disorder, such as AD using such compositions. The present invention is further directed to methods for identifying BACE1 modulators, candidate compounds that are BACE1 modulators, as well as, methods for treating, preventing, or ameliorating neurodegenerative diseases, such as AD, using such candidate compounds or pharmaceutical compositions containing such candidate compounds.

### DEFINITIONS

The phrase “High Throughput Screening” (HTS) as used herein defines a process in which large numbers of candidate compounds are tested rapidly and in parallel for binding activity or biological activity against target molecules. In HTS, the candidate compounds may act as, for example but not limited to, inhibitors of target enzymes, as competitors for binding of a natural ligand to its receptor, or as agonists/antagonists for receptor-mediated intracellular processes. In certain embodiments, large numbers of candidate compounds may be, for example, more than 100 or more than 300 or more than 500 or more than 1,000 candidate compounds. Preferably, the process is an automated process. HTS is a known method of screening to those skilled in the art.

In the present invention, virtual high throughput screening may be used. The phrase “virtual high throughput screening” (virtual HTS), as used herein, means a rapid filtering of large databases or libraries of candidate compounds though the use of computational approaches based on discrimination functions that permit the selection of candidate compounds to be tested for biological activity. Such approaches are within the skill of the art. See, e.g., Plewczyski et al., Chem. Biol. Drug. Res., 69(4):269-79 (2007), Lu et al., J. Med. Chem., 49(17):5154-61 (2006), Nicolazzo et al., J. Pharm. Pharmacol., 58(3):281-93 (2006), and Langer and Wolber, Pure Appl. Chem., 76(5):991-996 (2004).

In one HTS method according to the present invention, candidate compounds, such as candidate BACE1-modulators (e.g. BACE1 inhibitors, are selected in, e.g., a cell-based screening assay, such as, e.g., the cell-based BACE1 assay, described in further detail in Example 8 coupled with an ELISA assay, such as, e.g., the ELISA-SEAP assay described in further detail in Example 6. In such a method, candidate compounds are selected that have, for example, a % DMSO control greater than 2 standard deviations (SD) outside the mean. In the present invention, the cutoff value of 2 SD may be varied, from, e.g., greater than 1.25 SD to greater than 2.5, 3, 4, or 5 SD.

In the present invention, the phrase “cell-based BACE1 assay” means any suitable assay, preferably an HTS assay, which measures a candidate compound's ability to modulate BACE1 activity. A non-limiting example of such an assay includes the cell-based BACE1 assay described in Example 8.

In the present invention, SH-SY5Y cells were transfected and used in the cell-based BACE1 assay. The non-transformed cell line was obtained from ATCC (ATCC No. CRL-2266) (Manassas, Va.). The SH-SY5Y cell line, however, is available through a variety of publicly available sources, such as for example DSMZ (ACC No. 209), ECACC (ECACC No. 94030304), and ICLC (Accession No. ICLC HTL95013). Moreover, other appropriate cell lines, preferably human neuronal cell lines, that are capable of being stably transformed with the polycistronic vectors of the present invention, e.g., pBudCE4.1/BACEGFP-SEAPAPPwt, and functioning in the cell-based BACE1 assay may also be used.

As used herein, “ELISA assay” means any assay, preferably an HTS assay, which may be used to identify candidate compounds that modulate, bind to and/or inhibit BACE1. A non-limiting example of an ELISA assay is disclosed in Example 6. Although the assay in Example 6 is described in terms of a 96-well plate, the assay is easily adapted to 384-, or 1536-, or more well formats.

In the present invention, “solid supports” include, but are not limited to, substrates such as nitrocellulose (e.g., in membrane or microtitre well form); polyvinylchloride (e.g., sheets or microtitre wells); polystyrene latex (e.g., beads or microtitre plates); polyvinylidine fluoride; diazotized paper; nylon membranes; activated beads, magnetically responsive beads, and the like. Particular supports include plates (e.g., multi-well plates), arrays, microarrays, antibody chips, pellets, disks, capillaries, hollow fibers, needles, pins, solid fibers, cellulose beads, pore-glass beads, silica gels, polystyrene beads optionally cross-linked with divinylbenzene, grafted co-poly beads, polyacrylamide beads, latex beads, dimethylacrylamide beads optionally crosslinked with N-N′-bis-acryloylethylenediamine, and glass particles coated with a hydrophobic polymer.

If desired, the molecules, e.g., antibodies of the present invention, to be immobilized to the solid support can readily be functionalized to create styrene or acrylate moieties, thus enabling the incorporation of the molecules into polystyrene, polyacrylate or other polymers such as polyimide, polyacrylamide, polyethylene, polyvinyl, polydiacetylene, polyphenylene-vinylene, polypeptide, polysaccharide, polysulfone, polypyrrole, polyimidazole, polythiophene, polyether, epoxies, silica glass, silica gel, siloxane, polyphosphate, hydrogel, agarose, cellulose, and the like. In the present invention, any conventional method may be used to attach the antibodies to the solid support, e.g., 96-well plates. Such methods may be found, e.g., in U.S. Pat. No. 7,241,879, which is incorporated by reference as if recited in full herein.

The antibodies of the present invention may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the antibodies of the present invention and functional groups on the support or may be a linkage by way of a cross-linking agent). Immobilization by adsorption to a well in a microtitre plate or to a membrane is preferred. In such cases, adsorption may be achieved by contacting the antibodies of the present invention, in a suitable buffer, with the solid support for a suitable amount of time. The contact time varies with temperature, but is typically between about 1 hour and about 1 day. In general, contacting a well of a plastic microtitre plate (such as polystyrene or polyvinylchloride) with an amount of an antibody of the present invention ranging from about 10 ng to about 10 μg, and preferably about 100 ng to about 1 μg, is sufficient to immobilize an adequate amount of antibody.

Covalent attachment of an antibody according to the present invention to a solid support may generally be achieved by first reacting the support with a bifunctional reagent that will react with both the support and a functional group, such as a hydroxyl or amino group, on the antibody. For example, the antibody may be covalently attached to supports having an appropriate polymer coating using benzoquinone or by condensation of an aldehyde group on the support with an amine and an active hydrogen on the antibody (see, e.g., Pierce Immunotechnology Catalog and Handbook, 1991, at A12-A13).

In certain embodiments, the screening methods may be performed by first contacting an antibody that has been immobilized on a solid support, commonly the well of a microtitre plate, with the sample, such that polypeptides, e.g., sAPPβ within the sample are allowed to bind to the immobilized antibody. Unbound sample is then removed from the immobilized polypeptide-antibody complexes and a detection reagent (preferably a second antibody as in Methods 1A and 1B, capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support may be blocked. Any suitable blocking agent known to those of ordinary skill in the art, such as bovine serum albumin or Tween 20®. (Sigma Chemical Co., St, Louis, Mo.) may be used. The immobilized antibody may then be incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual. In one embodiment, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At 37° C., an incubation time of about 30 minutes to about 10 hours may generally be sufficient.

Unbound sample may then be removed by washing the solid support with an appropriate buffer, such as PBS containing 0.1% Tween 20®. The second antibody, which contains a reporter group, may then be added to the solid support.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups, Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

The terms “about” or “approximately” mean within an acceptable error range for the particular value as determined by one of ordinary skill in the art, which will depend, in part, on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably up to 1%, 2%, 3%, or 4% of a given value. Alternatively, particularly with respect to biological systems or processes, the terms can mean within an order of magnitude, preferably within 5-fold, and more preferably within 2-fold, of a value.

As used herein, “modulates BACE1 activity” (or levels) refers to the ability of a molecule to change, e.g., inhibit or decrease, e.g., BACE1 cleavage of APP in cells, such as neurons, thereby promoting cell viability (growth or proliferation) by decreasing or alleviating the build up of sAPPβ. Preferably, “modulates BACE1 activity” or levels refers to the function of candidate compounds identified in one of the screening assays of the present invention, which function may be catalytic or allosteric inhibition of BACE1. BACE1 modulators according to the present invention may also act indirectly on BACE1 by, e.g., modulating trafficking and/or accessibility of APP and/or BACE1. Preferably, the BACE1 modulators identified in one of the screening assays of the present invention are selective for APP over other BACE1 substrates. Large-scale screens include screens wherein hundreds or thousands or more of candidate compounds are screened in a high-throughput format for BACE1 modulators and inhibitors in neuronal cells.

As used herein, “neurodegeneration” or a “neurodegenerative disease” refers to a disease state characterized by progressive loss of neural function. In the present invention, such disease states include, AD, early onset familial AD, amyotrophic lateral sclerosis (Lou Gehrig's Disease), Binswanger's Disease, corticobasal degeneration (CBD), dementia lacking distinctive histopathology (DLDH), frontotemporal dementia (FTD), Huntington's chorea, multiple sclerosis, myasthenia gravis, Parkinson's disease, and progressive supranuclear palsy (PSP). Preferably, the neurodegenerative disorder is AD, such as for example, early onset familial AD.

The term “treat” is used herein to mean to relieve or alleviate or delay the progression of at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

The term “prevent” or prevention is used in terms of prophylactic administration of a compound or pharmaceutical composition prior to the onset of disease or to prevent recurrence of a disease. Administration of the dosage form to prevent the disease need not absolutely preclude the development of symptoms. Prevent can also mean to reduce the severity of the disease or its symptoms.

The phrase “pharmaceutically acceptable” as used in connection with compounds and compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

As used herein, “candidate compounds” encompass numerous chemical classes, although typically they are organic molecules, preferably small organic compounds having a molecular weight of more than 50 and less than about 2,500 daltons. Candidate compounds comprise functional groups necessary for structural interaction with proteins, particularly hydrogen bonding, and typically include at least an amine, carbonyl, hydroxyl or carboxyl group, preferably at least two of these functional chemical groups. The candidate compounds may also comprise cyclical carbon or heterocyclic structures and/or aromatic or polyaromatic structures substituted with one or more of the above functional groups. Candidate compounds are also found among biomolecules including but not limited to peptides, saccharides, fatty acids, steroids, purines, pyrimidines, derivatives, structural analogs or combinations thereof.

Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

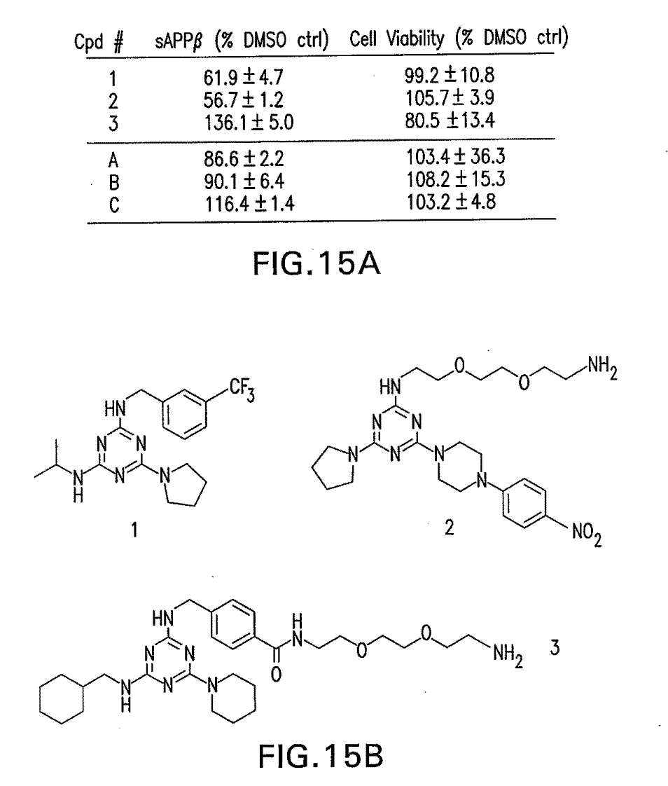

In the present invention, “BACE1 modulator” means an agent, such as for example, a small molecule, that effects BACE1 activity, e.g., inhibits BACE1 activity and modulates neurodegeneration in a subject or patient such as a mammal, e.g., a human or an animal. BACE1 inhibitors include both catalytic and allosteric inhibitors of BACE1. Preferably, such inhibitors are specific for APP over other BACE1 substrates. BACE1 inhibitors may be identified using the methods disclosed herein. Representative examples of candidate compounds that have a significant effect on sAPPβ are BACE1 modulators, including Compounds 1-3 shown in FIG. 15B. In the present invention, where appropriate, the “BACE1 modulators,” including BACE1 inhibitors, embrace analogs, enantiomers, optical isomers, diastereomers, N-oxides, crystalline forms, hydrates, pharmaceutically acceptable salts, and combinations of the compounds disclosed herein. The present invention also includes pharmaceutical compositions containing one or more of these compounds.

The compounds and compositions of the present invention may be administered in any appropriate manner. For example, candidate compounds can be profiled in order to determine their suitability for inclusion in a pharmaceutical composition. One common measure for such agents is the therapeutic index, which is the ratio of the therapeutic dose to a toxic dose. The thresholds for therapeutic dose (efficacy) and toxic dose can be adjusted as appropriate (e.g., the necessity of a therapeutic response or the need to minimize a toxic response). For example, a therapeutic dose can be the therapeutically effective amount of a candidate compound (relative to treating one or more conditions) and a toxic dose can be a dose that causes death (e.g., an LD₅₀) or causes an undesired effect in a proportion of the treated population. Preferably, the therapeutic index of a compound, agent, or composition according to the present invention is at least 2, more preferably at least 5, and even more preferably at least 10. Profiling a candidate compound can also include measuring the pharmacokinetics of the compound, to determine its bioavailability and/or absorption when administered in various formulations and/or via various routes.

A candidate compound of the present invention, such as a compound that modulates BACE1 activity, e.g., a BACE1 inhibitor, may be administered to an individual in need thereof. In certain embodiments, the individual is a mammal such as a human, or a non-human mammal. When administered to an individual (subject), the compound of the invention can be administered as a pharmaceutical composition containing, for example, the compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a preferred embodiment, when such pharmaceutical compositions are for human administration, the aqueous solution is pyrogen free, or substantially pyrogen free. Excipients may be selected and incorporated into such compositions, for example, to effect delayed release of an agent or to selectively target one or more cells, tissues or organs.

A pharmaceutically acceptable carrier can contain physiologically acceptable agents that act, for example, to stabilize or to increase the absorption of a compound, such as, a BACE1 modulator. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the invention. Liposomes, for example, which consist of phospholipids or other lipids, are nontoxic, physiologically acceptable and metabolizable carriers that are relatively simple to make and administer.

### Administration

A pharmaceutical composition (preparation) containing a compound of the invention can be administered to an individual by any of a number of routes of administration including, for example, orally; intramuscularly; intravenously; anally; vaginally; parenterally; nasally; intraperitoneally; subcutaneously; and topically. The composition can be administered by injection or by incubation.

In certain embodiments, the compound (e.g., BACE1 modulator) of the present invention may be used alone or conjointly administered with another type of agent designed to mediate neurodegeneration. As used herein, the phrase “conjoint administration” refers to any form of administration in combination of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

It is contemplated that the compounds (e.g., BACE1 modulators) of the present invention will be administered to an individual (e.g., a mammal, preferably a human) in a therapeutically effective amount (dose). By “therapeutically effective amount” is meant the concentration of a compound that is sufficient to elicit the desired therapeutic effect (e.g., treatment of a condition, the accumulation of Aβ). It is generally understood that the effective amount of the compound will vary according to the weight, sex, age, and medical history of the individual. Other factors which influence the effective amount may include, but are not limited to, the severity of the individual's condition, the disorder being treated, the stability of the compound, and, if desired, another type of therapeutic agent being administered with the compound of the invention. Typically, for a human subject, an effective amount will range from about 0.001 mg/kg of body weight to about 50 mg/kg of body weight. A larger total dose can be delivered by multiple administrations of the agent. Methods to determine efficacy and dosage are known to those skilled in the art. See, for example, Isselbacher et at. (1996) Harrison's Principles of Internal Medicine 13 ed., 1814-1882, herein incorporated by reference.

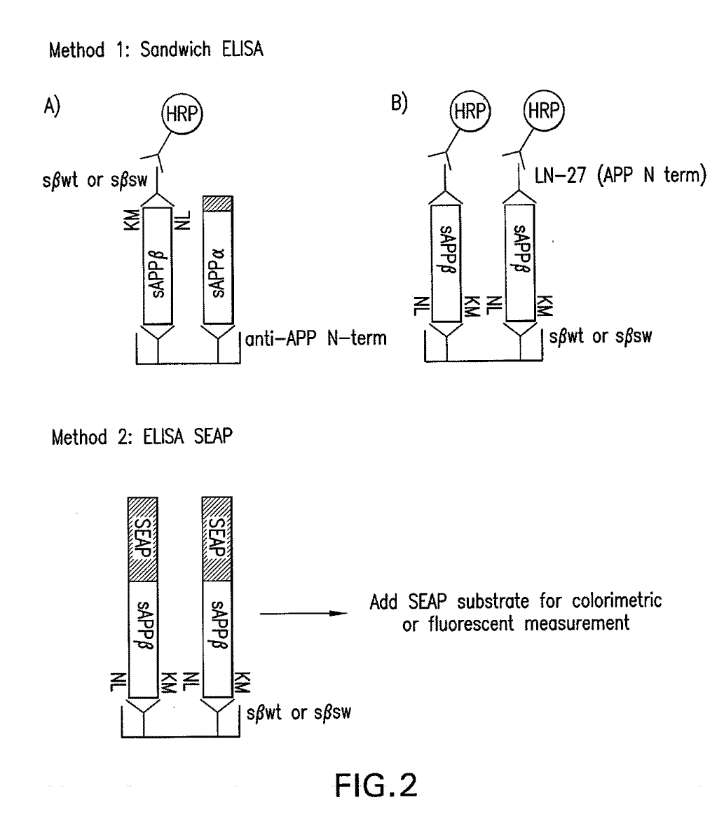

In the present invention, a first method is provided, which is a sandwich ELISA using an APP N-terminal antibody for capture and a β-site-specific antibody for detection (FIG. 2: Method 1A). A reciprocal assay is also provided using a β-site-specific antibody for capture and an APP N-terminal antibody for detection (FIG. 2: Method 1B). A further method is provided, which is a hybrid ELISA-SEAP assay that takes advantage of an APP construct containing an N-terminal secreted alkaline phosphatase (SEAP) domain, (FIG. 2: Method 2)

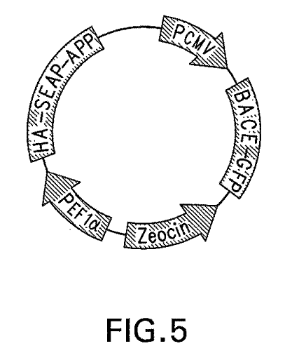

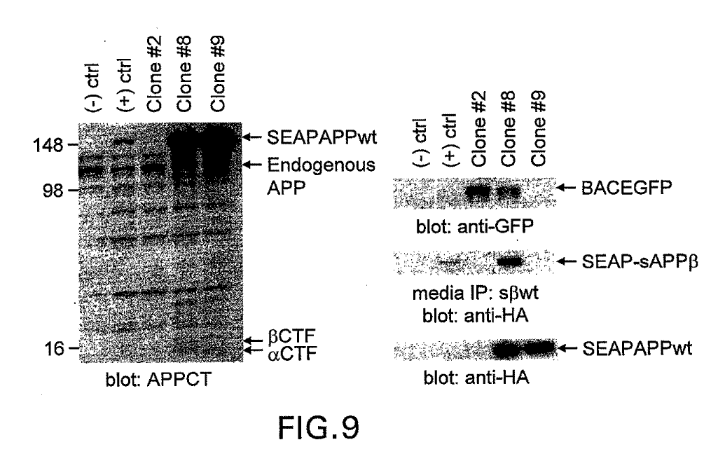

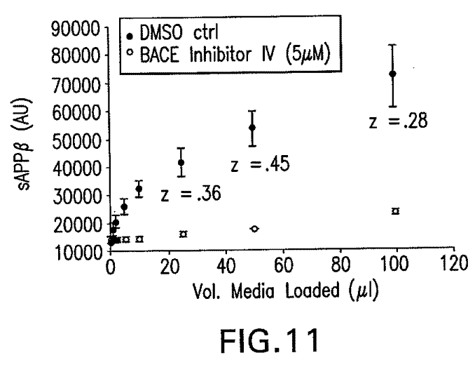

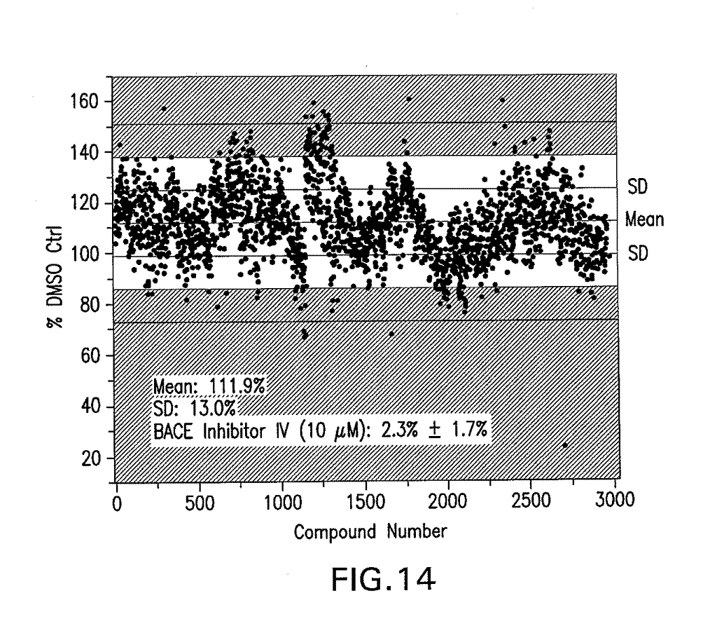

A cell-based screening assay (cell-based BACE1 assay) has also been developed to compliment the ELISA-SEAP assay. In particular, a double stable cell line expressing BACE1-GFP and SEAP-APPwt (made using the bicistronic vector, FIG. 5) has been generated (FIG. 9), which produces robust sAPPβ in the media for measurement (FIG. 11). The cell-based BACE1 assay has been rigorously validated (FIGS. 11-13), and has been used in a chemical screening application using a tagged-triazine library from Dr. Young-Tae Chang (Library Reference Khersonsky S M et al., Journal of the American Chemical Society, Vol, 125, pp. 11804-11805 (2003), which is incorporated by reference as if recited in full herein. (FIGS. 14-15). Three candidate compounds from the tagged-triazine library have been identified that down regulate or upregulate sAPPβ generation using the cell-based screening assay (FIG. 15).

The cell-based BACE1 assay offers numerous advantages. Unlike FRET-based cell-free BACE1 assays, the cell-based BACE1 assay has the potential to discover indirect small molecule modulators of BACE1 that are effective in intact cells, in addition to identifying direct BACE1 inhibitors. For instance, the cell-based BACE1 assay has the ability to identify small molecules that can modulate the trafficking and/or accessibility of APP and BACE1, and that function as allosteric regulators. Thus, small molecule tools that probe various aspects of BACE1 biology (i.e., metabolism and function) can be identified.

Furthermore, the cell-based BACE1 assay has the ability to monitor BACE1-mediated cleavage of APP, a canonical pathway in AD pathogenesis. Numerous alternative substrates have been identified for BACE1 (e.g., neuregulin). The cell-based BACE1 assay, however, can identify BACE1 modulators that are selective for APP over other BACE1 substrates. Moreover, the cell-based BACE1 assay uses wild type forms of APP, which may allow for identification of small molecule modulators that function with native APP molecules. In addition, the cell-based BACE1 assay strategy generally helps to overcome the drawbacks associated with peptide-substrate-based cell-free protease assays (e.g., poor cell-permeability and cytotoxicity).

In sum, the methods and compositions of the present invention provide convenient means to detect and/or quantify BACE1 activity, identify candidate compounds that are BACE1 modulators, and use such compounds to treat, prevent, or alleviate the symptoms of, e.g., BACE1-mediated neurodegeneration. The screening methods and compositions provide a powerful platform on which small molecule and RNA screening—both conventional and high-throughput—can take place to not only identify potential therapeutic candidates (candidate compounds) for, e.g., AD, but also to elucidate the regulation of BACE1.

Because the methods of the present invention are able to identify and quantitate sAPPβ secreted into the extracellular space, use of these methods to quantify sAPPβ in biological samples, such as human cerebrospinal fluid (CSF), may provide novel diagnostic tests for neurodegenerative disorders, including AD.

The following examples are provided to further illustrate the compositions and methods of the present invention. These examples are illustrative only and are not intended to limit the scope of the invention in any way.

### EXAMPLES

### Example 1

### β-Site Cleavage-Specific Antibodies Against Wild-Type or Swedish Mutant Form of β-Amyloid Precursor Protein (APP)

To selectively detect soluble APP derived from β-secretase-mediated cleavage, two separate peptides were synthesized based on the human sequence. Each peptide comprised the β-secretase cleavage site of either wild-type or Swedish FAD variants of APP. The sequence of each peptide is set forth below:

| sAPPβwt: | |
|---|---|
| (C)GGGISEVKM-COOH; | (SEQ ID NO: 1) |
| sAPPβsw: | |
| (C)GGGISEVNL-COOH. | (SEQ ID NO: 2) |

Using standard protocols, the peptides were conjugated with keyhole limpet hemocyanin (KLH) and subsequently used to immunize rabbits to generate the sAPPβwt and sAPPβsw antibodies sβwt and sβsw. If desired, the antibodies may be further IgG purified. Particularly with reference to the ELISA-SEAP, it is preferred to use IgG purified sβwt antibody for capture. IgG purification was carried out using standard procedures. An example of such a standard procedure may be found, e.g., in Sambrook et al., Molecular Cloning—A Laboratory Manual, 2^{nd} Ed., vol. 3, pp. 18.11-18.13 (1989), which is incorporated by reference as if recited in full herein.

### Example 2

### sAPPβ Detection Assays

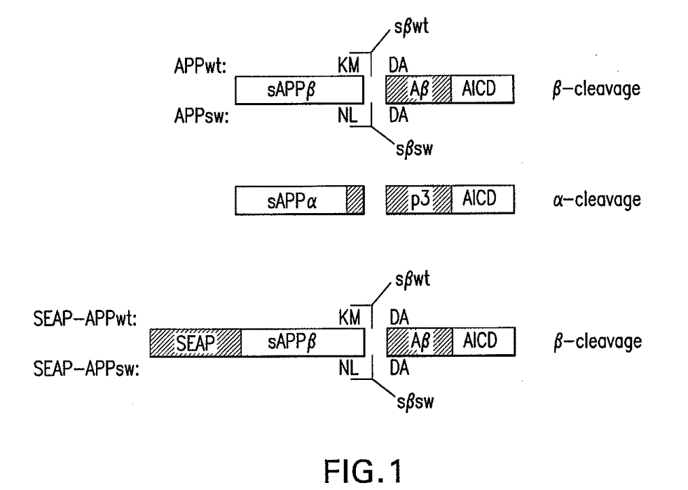

In the present invention, novel detection methods are disclosed, which are based on antibodies that are specific to sAPPβ (FIG. 1) and discriminate against the α-secretase-cleaved forms of secreted APP (sAPPα).

As shown in FIG. 2, a first method (1A) is depicted as a sandwich ELISA using an APP N-terminal antibody for capture and the β-site-specific antibodies of the present invention for detection. The labelled third antibodies used to detect the sandwich are goat α-mouse, horseradish peroxidase labelled antibodies. The labelled antibodies and antibody coated plates were obtained from BioSource. The present invention also includes the reciprocal assay (Method 1B in FIG. 2) which uses the β-site-specific antibodies for capture and an APP N-terminal antibody for detection.

As also shown in FIG. 2, another method is depicted as a hybrid ELISA-SEAP assay that takes advantage of an APP construct containing an N-terminal secreted alkaline phosphatase (SEAP) domain (FIG. 1). In this method, the β-site-specific antibodies of the present invention are used to capture SEAP-tagged sAPPβ in cell culture media. Following capture, a substrate is added and alkaline phosphatase substrate metabolism is used to easily detect and/or quantify sAPPβ by colorimetric or fluorescent means. One preferred substrate is 4-methylumbelliferyl phosphate (4-MUP) obtained from Sigma (St. Louis, Mo.).

### Example 3

### Specificity of sAPPβ antibodies

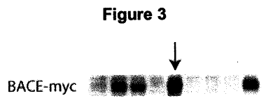

Human BACE1 (the polynucleotide and polypeptide sequences of which are well known, see, e.g., (4) and (14)) was subcloned into pcDNA3.1/myc-His vector (Invitrogen) containing a neomycin resistance gene. Commercially available mouse neuroblastoma Neuro2a native cells (such as, for example, ATCC No. CCL-131) were transfected with the BACE-myc construct and selected with G418 (Calbiochem) at 1 mg/ml concentration. Nine colonies were selected (FIG. 3) and probed with the anti-myc antibody 9E10 (Covance) on Western blot analysis. The colony with the highest BACE1 expression (arrow) was selected as the Neuro2a-BACE stable cell line.

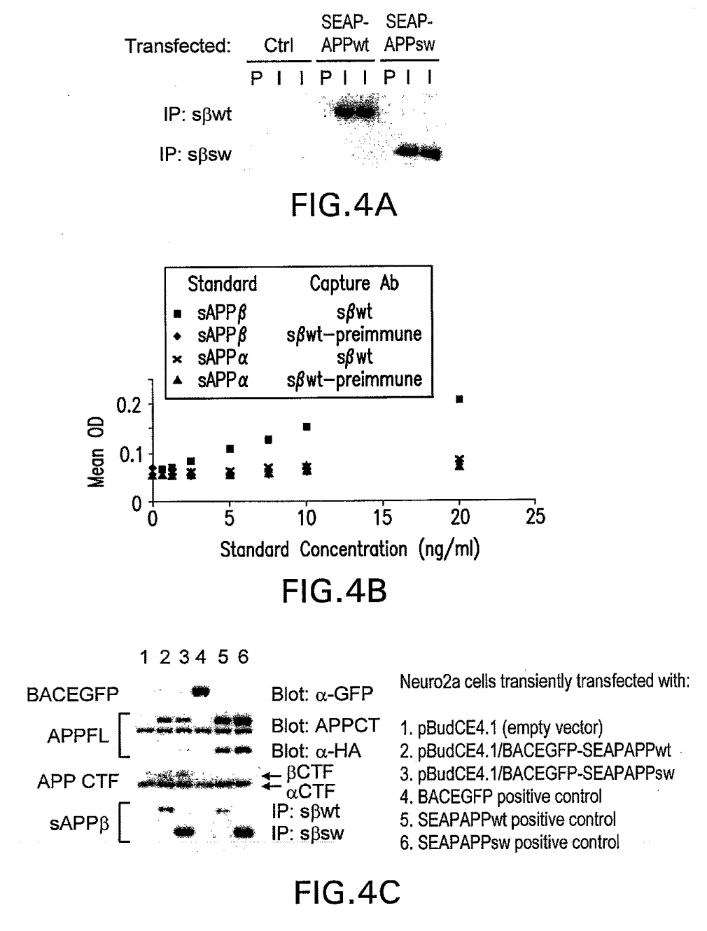

Neuro2a-BACE cells were transiently transfected with empty vector, SEAP-APPwt, or SEAP-APPsw. Culture media was immunoprecipitated with pre-immune serum (P) or the sAPPβ antibody (I) and visualized on Western blot with anti-HA because SEAP-APP constructs also contain an N-terminal HA tag. sβwt reacts only to sAPPwt and not to sAPPβsw. In addition, sβsw does not cross-react with sAPPβwt. (FIG. 4A).

sAPPβ ELISA (Method 1B) was performed using recombinant sAPPα and sAPPβ (Sigma). The pure peptides were captured with sβwt or pre-immune serum and detected indirectly with an unlabelled LN-27 (a mouse anti-human IgG₁ from Zymed) followed by a labelled second antibody, such as an HRP-conjugated goat anti-mouse IgG (from Pierce). sβwt did not capture sAPPα. (FIG. 4B). Alternatively, the pure peptides may be captured with sβwt or pre-immune serum and detected directly with, e.g., a labelled pan-APP antibody LN-27.

Two cDNA expression plasmids encoding BACEGFP plus SEAP-APPwt or BACEGFP plus SEAP-APPsw were generated using the bicistronic vector pBudCE4.1 (Invitrogen) according to the manufacturer's instructions. These constructs allow simultaneous overexpression of BACE and SEAP-APP.

Briefly, the bicistronic vectors pBudCE4.1/BACEGFP-SEAPAPPwt and pBudCE4.1/BACEGFP-SEAPAPPsw were made as follows: BACE1 ORF from the BACE-myc plasmid was subcloned into the pEGFP-N1 vector (Clontech) using Xho I and BamHI restriction sites. The PCR primers used for the reaction were the T7 sequencing primer and TAGTAGCGAGGATCCAGCTTCAGCAGGGAGATG (SEQ ID NO:3).

The SEAP-APP construct was subcloned into the peak12 vector containing an EF-1α promoter and a puromycin resistance gene using NotI and HindIII restriction sites. peak12/(HA)SEAP-APP constructs (“SEAPAPPwt” and “SEAPAPPsw”) were a gift of Dr. Stefan Lichtenthaler (Adolf-Butenandt-Institut, Ludwig-Maximilians-University, Munich, Germany).

The bicistronic cloning vector pBudCE4.1 was purchased from Invitrogen. BACEGFP was cloned into the HindIII and XbaI sites in the P_{CMV} multicloning site using the primers:

(SEQ ID NO: 4)
TCATTGAAGCTTATGGCCCAAGCCCTGC
and
(SEQ ID NO: 5)
TAGCGATCTAGATTACTTGTACAGCTCGTCCATGCC.
(See, FIG. 5).

SEAPAPPwt and SEAPAPPsw were cloned into the Not I and Icon I sites of the P_{EF-1α} MCS using the primers:

| | |
|---|---|
| TCATTCGCGGCCGCCTAGCTAGAGATCCCTCG | (SEQ ID NO: 6) |
| and | |
| TAGCGAGGTACCGGCCGCTTAGTTCTGCAT. | (SEQ ID NO: 7) |
| (See, FIG. 5). | |

BACE1 was visualized with the anti-GFP antibody JL-8 (BD Biosciences), full-length APP (APPFL) and APP C-terminal fragments (APP CTF) were visualized with APPCT, a polyclonal antibody directed against the C-terminus of APP. APPCT is a polyclonal antibody produced using standard methods by immunizing rabbits with KLH-conjugated peptides corresponding to the C-terminal region of APP: (C)HLSKMQQNGYENPTYKFFEQMQN (SEQ ID NO:8). APPFL was also probed with anti-HA. sAPPβ was obtained using the same protocol described above. Simultaneous overexpression of BACE and APP resulted in increased β-cleavage compared to overexpression of APP alone. This is evidenced by increased sAPPβ levels and increased βCTF production. (FIG. 4C)

### Example 4

### sAPPβ Sandwich ELISA Employing App N-Terminal Capture Antibody and sAPPβ-Specific Detection Antibody (Method IA)

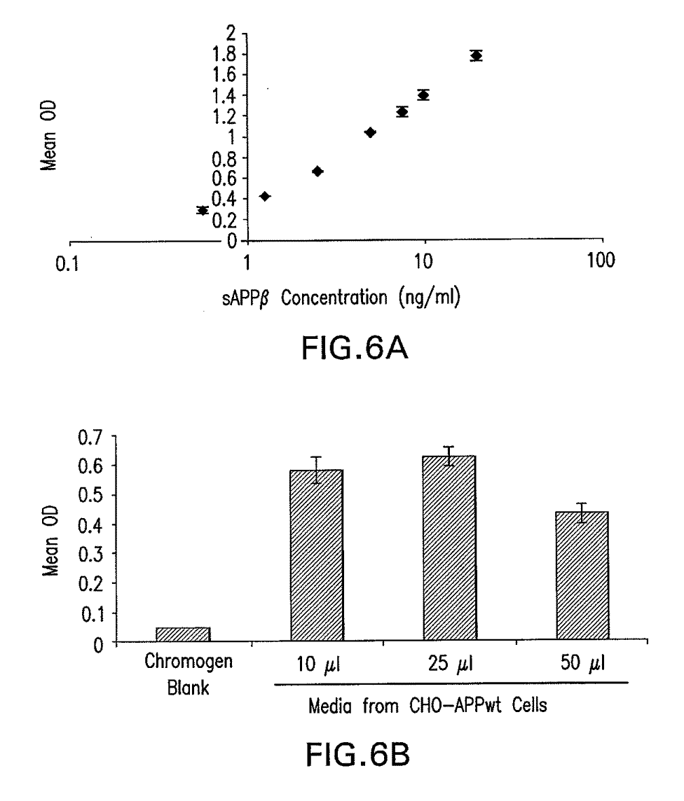

A standard curve using purified human sAPPβ was generated. (FIG. 6A)

CHO-APPwt cells were grown to 80% confluence in 6-well culture plates. Cells were incubated in 700 μl of culture media for 24 hours and 10, 25, or 50 μl each of the media was loaded onto an ELISA assay plate, diluted in 90, 75, and 50 μl diluent buffer, respectively. Increasing the volume of media loaded does not result in increased sAPPβ signals. (FIG. 6B). This is likely due to increased competition for capture antibody binding by sAPPα, which is present at significantly higher concentrations than sAPPβ. It is plausible that sAPPβ can still be detected at a lower concentration range.

### Example 5

### sAPPβ Sandwich ELISA Employing sAPPβ-Specific Capture Antibodies and APP N-Terminal Detection Antibody (Method 1B)

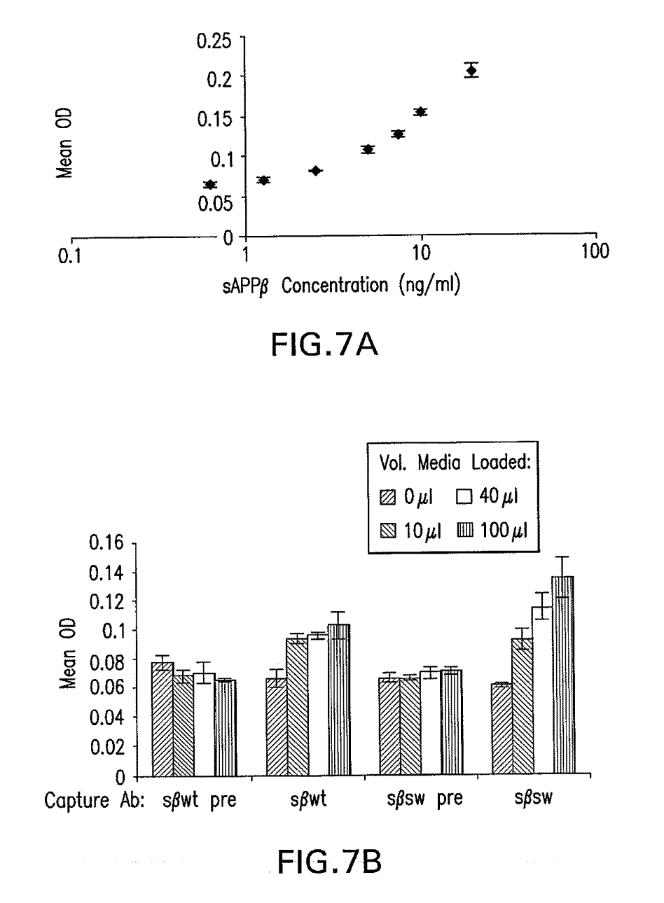

Purified recombinant human sAPPβ peptide (Sigma) was used to generate a standard curve. (FIG. 7A). Method 1B was also used to verify the specificity of β-site cleavage-specific antibodies (FIG. 4B).

With reference to FIG. 7B, the indicated volume of culture media from Neuro2a cells transiently transfected with pBudCE4.1/BACEGFP-SEAPAPPwt or sw was loaded onto a 96-well plate coated with the indicated capture antibodies. The captured sAPPβ was detected with anti-HA antibody recognizing the HA tag at the N-terminus of SEAPAPP. As shown in FIG. 7B, sβwt and sβsw captured their respective sAPPβ's.

### Example 6

### Hybrid ELISA-SEAP Assay for sAPPβ Detection (Method 2)

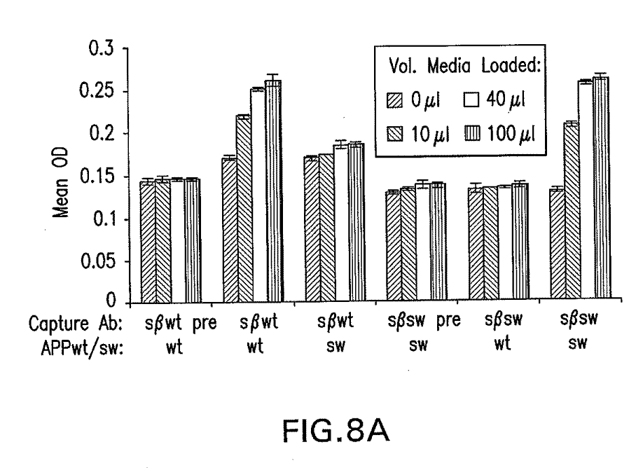

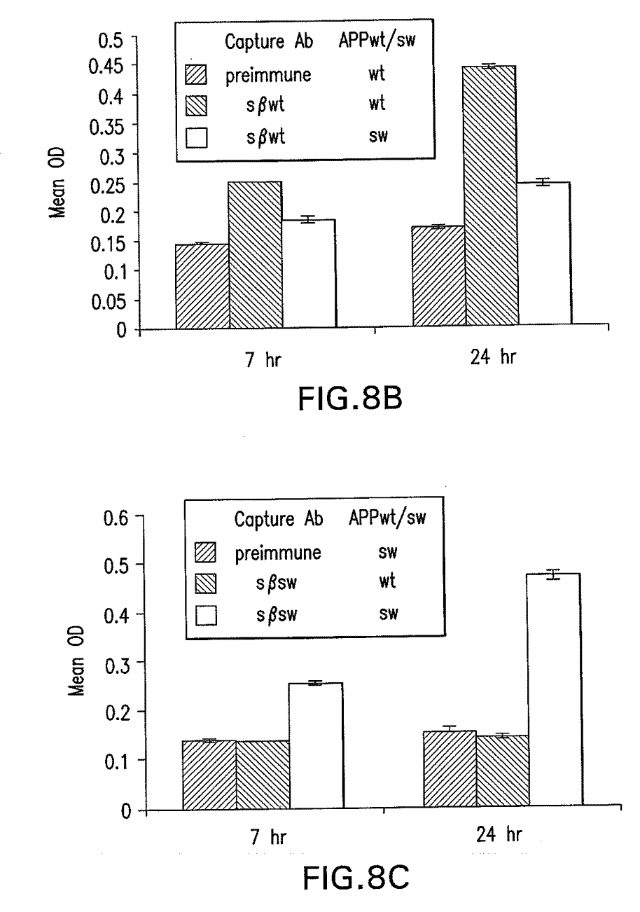

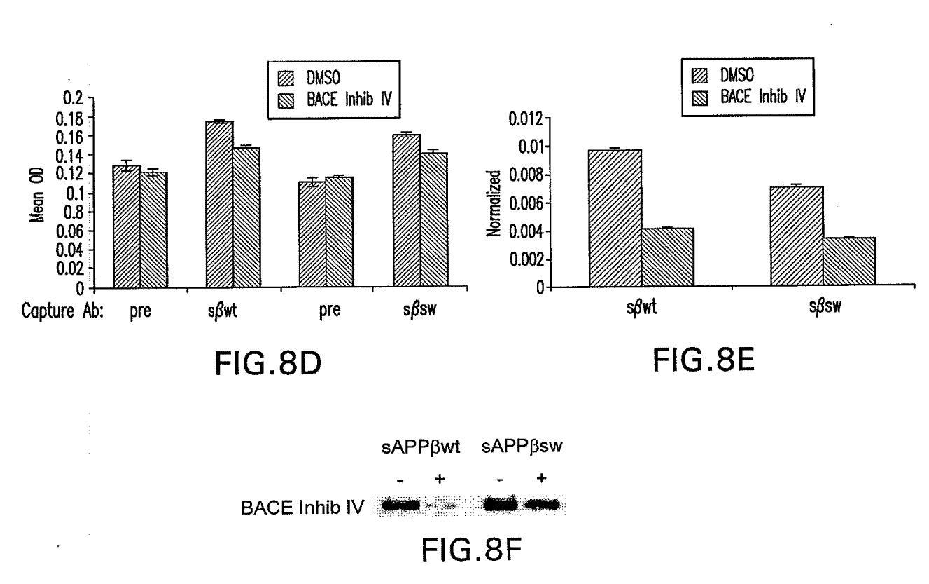

With reference to FIG. 8A, the indicated volume of culture media from Neuro2a cells transiently transfected with pBudCE4.1/BACEGFP-SEAPAPPwt or sw was loaded onto a 96-well plate coated with the indicated capture antibodies. The captured sAPPβ was incubated with the SEAP substrate, 1-step pNPP (Pierce), for 7 hr at 37° C. As shown in FIG. 8A, sβwt and sβsw captured their respective sAPPβ's. There is some cross-reactivity of sβwt for sAPPβsw, but there is no cross-reactivity of sβsw for sAPPβwt.

As shown in FIGS. 8B and 8C, the same samples from FIG. 8A were allowed to incubate overnight with the SEAP substrate. The OD values were read at 24 hr, and the 40 μl volume data was plotted against incubation time. As shown in FIGS. 8B and 8C, the SEAP signal showed a large increase over time, while the background (pre-immune) increased at a much lower rate.

Neuro2a cells transiently transfected with pBudCE4.1/BACEGFP-SEAPAPPwt or sw were subjected to treatment by 5 μM BACE inhibitor IV (Calbiochem) for 6 hr. 40 μl media was loaded onto wells coated with the indicated capture antibodies and subjected to 6 hr SEAP substrate incubation. FIG. 8D shows the raw data from this experiment and FIG. 8E shows the background-subtracted data, normalized to total protein. BACE inhibitor IV reliably reduced sAPPβ levels in both cells transfected with APPwt and those transfected with APPsw,

Media from the experiments shown in FIGS. 8D and 8E was immunoprecipitated with either sβwt or sβsw and visualized with anti-HA by Western blot analysis. The results of this experiment are shown in FIG. 8F.

### Example 7

### Characterization of SY5Y-BACEGFP-SEAPAPPwt Stable Cells

To facilitate stable cell line generation, BACEGFP and SEAPAPPwt were first subcloned into a single bicistronic vector (pBudCE4.1, Invitrogen, data not shown) according to the manufacturer's protocol. Cells from the human neuroblastoma cell line SH-SY5Y available through, e.g., ATCC (ATCC No. CRL-2266), were transfected with the bicistronic vector containing BACEGFP and SEAPAPPwt and selected with Zeocin. Three clones (#2, #8, and #9) were selected for testing. Only clone #8 expresses both BACEGFP and SEAPAPPwt. (−) ctrl represents native SY5Y cells and (+) ctrl represents SY5Y cells transiently transfected with pBudCE4.1/BACEGFP-SEAPAPPwt. Characterization of the SY5Y-BACEGFP-SEAPAPPwt stable cells is shown in FIG. 9.

### Example 8

### Cell-Based BACE1 Assay

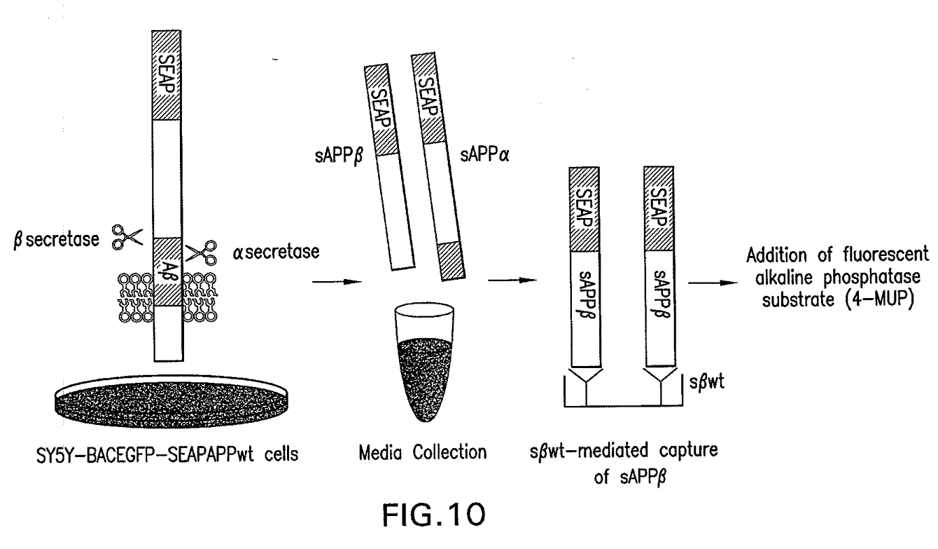

SY5Y cells expressing BACEGFP and SEAPAPPwt were grown on standard cell culture plates in DMEM (Invitrogen) supplemented with 10% fetal bovine serum, 1% Pen/Strep/Glutamine, and 250 μg/ml zeocin. SEAPAPPwt is cleaved by BACE1 (β-secretase) as well as a secretase to release large extracellular fragments sAPPβ and sAPPα, respectively. Cell culture media containing these fragments was harvested and SEAP-sAPPβ was captured using 96-well plates coated with the sAPPβ specific antibody (sβwt). The ELISA plate was then incubated with the fluorescent alkaline phosphatase substrate 4-MUP (Sigma) at a final concentration of 30 μM (in 100 mM glycine, 1 mM MgCl₂, 1 mM ZnCl₂ at pH 9.8) for 1 hour before detection (excitation: 360 nm, emission: 449 nm). A schematic of this cell-based BACE1 assay is shown in FIG. 10

### Example 9

### Validating the Cell-Based BACE1 Assay

### BACE1 Assay—A Viable HTS

SY5Y-BACEGFP-SEAPAPPwt stable cells were grown to 100% confluence on 6-well plates and incubated for 6 hours with either DMSO or 5 mM BACE inhibitor IV (CalBiochem) in 1 ml conditioned media. A sAPPβ ELISA-SEAP assay according to the method of Example 6 and as shown in FIG. 10 was then conducted. The addition of 5 mM BACE inhibitor IV abolished the SEAP signal with high z factors for the 3 data points calculated (FIG. 11). Z factor is defined as 1-(3(s⁺+s⁻)/(m⁺−m⁻, where “s” is the standard deviation and “m” is the mean value of the DMSO (+) and BACE inhibitor IV (−) data points. Z factors greater than 0.2 are considered acceptable for high throughput screening applications.

### The Cell-Based BACE1 Assay Validation Using Known Inhibitors

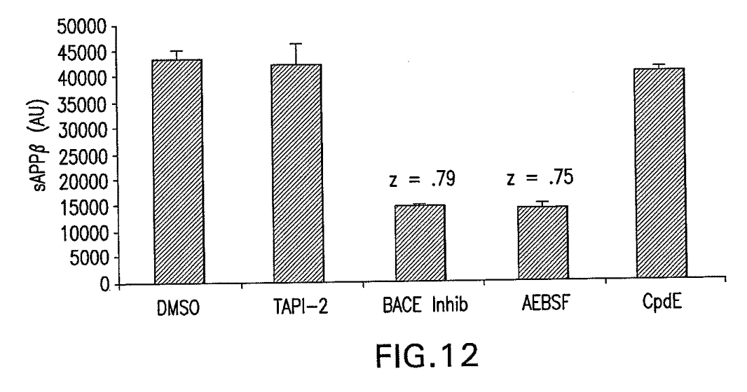

SY5Y-BACEGFP-SEAPAPPwt stable cells were grown to 100% confluence on 96-well plates in quadruplicate and incubated for 6 hours with DMSO, TAPE-2 (100 mM), BACE inhibitor IV (5 mM), AEBSF (500 mM), or Cpd E (20 nM) in 150 ml of conditioned media (DMEM (Invitrogen) supplemented with 10% fetal bovine serum, 1% Pen/Strep/Glutamine, and 250 μg/ml zeocin). The sAPPβ ELISA-SEAP assay was conducted using 50 ml of media. BACE Inhibitor IV and the serine protease inhibitor AEBSF dramatically reduced sAPPβ, while the α-secretase inhibitor TAPI-2 and the γ-secretase inhibitor CpdE had no effect (FIG. 12). The drug concentrations were selected based on previous experimental results. BACE Inhibitor IV and AEBSF are both effective at inhibiting their respective targets and do not exhibit significant cellular toxicity (data not shown).

### The Cell-Based BACE1 Assay—Dose Response Curve

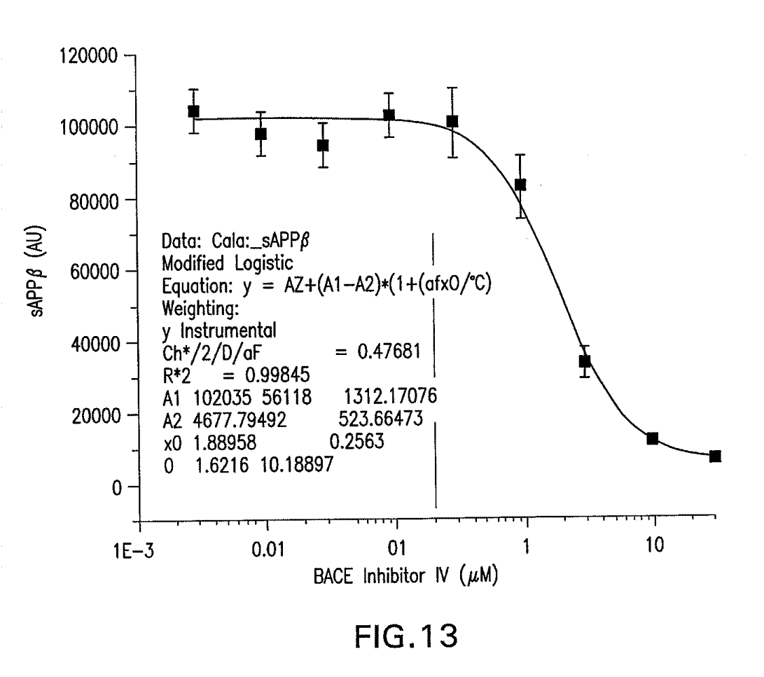

SY5Y-BACEGFP-SEAPAPPwt stable cells were grown to 100% confluence on 96-well plates in quadruplicate and incubated for 6 hours with the indicated concentrations of BACE Inhibitor IV in 150 ml of conditioned media (DMEM (Invitrogen) supplemented with 10% fetal bovine serum, 1% Pen/Strep/Glutamine, and 250 μg/ml zeocin) (FIG. 13). A sAPPβ ELISA-SEAP assay was conducted using 50 ml of media. The dose-response curve was fitted using a logistic model with Origin software, giving an IC₅₀ of 1.89 mM. The reported IC₅₀ are 15 nM (in vitro) and 29 nM in HEK293-APPNFEV cells.

### Example 10

### Primary Screen—Stage 1

Stage 1 of a primary screen was completed with a ˜3000-compound tagged triazine library. SY5Y-BACEGFP-SEAPAPPwt cells were grown to 100% confluence in 96-well plates and incubated with library compound (10 mM), 1:100 DMSO (triplicate), or 10 mM BACE Inhibitor IV (triplicate) in 150 ml of culture media (DMEM (Invitrogen) supplemented with 10% fetal bovine serum, 1% Pen/Strep/Glutamine, and 250 μg/ml zeocin). Media was collected after 6 hrs incubation and 50 ml was used in the BACE1 assay as described above. The library had a mean effect of 111.9%±13.0%, while the BACE1 inhibitor reduced SAPPβ to 2.3%±1.7% of DMSO control (FIG. 14), Candidate compounds that are >2 SD outside the mean (125 compounds) were selected for rescreening (triplicate).

### Example 11

### Primary Screen—Stage 2

In Stage 2, the 125 candidate compounds identified in Stage 1 of the primary screen were rescreened. Three candidate compounds had a significant effect on sAPPβ—Compounds 1, 2, and 3 (FIG. 15A, top). The structures of these compounds are set forth in FIG. 15B. The remainder of the rescreened candidate compounds either had less or no effect, or were significantly cytotoxic as measured by CellQuanti-Blue (BioAssay Systems). Three related but negative compounds are shown for comparison—Compounds A, B, and C (FIG. 15A, bottom).

In sum, unlike FRET-based cell-free BACE1 assays, the present cell based assays may be used to identify indirect small molecule modulators of BACE1 that are effective in intact cells in addition to identifying both catalytic and allosteric inhibitors of BACE1. Furthermore, the assays disclosed herein are designed specifically to monitor BACE1-mediated cleavage of APP, a canonical pathway in AD pathogenesis. Because numerous alternative substrates have been identified for BACE1 (e.g. neuregulin), the assays may identify BACE1 modulators that are selective for APP over other BACE1 substrates. Also because the present assays utilize wild type forms of APP, such assays may allow for identification of small molecule modulators that function with native APP molecules.

The cell-based BACE1 assay has been rigorously validated herein, and has been used by the inventors to identify three candidate compounds in a screen of an established tagged-triazine library (approximately 3000 compounds). Two of the candidate compounds reduced sAPPβ by 38% and 44%, respectively. The third candidate compound increased sAPPβ by 36%. All three candidate compounds have minimal effect on cell viability.

Successful medicinal chemistry on these candidate compounds may lead to novel therapeutic agents for the treatment of AD. Indeed, we expect that structure-activity-relationship studies on the three candidate compounds, and other compounds identified by the assays of the present invention, will produce potent analogs for therapeutic use as well as tools for identifying novel proteins targets involved in modulation of BACE1-mediated APP cleavage.

### CITED DOCUMENTS

The following documents, cited above, are incorporated by reference as if recited in full herein:
1. Selkoe D J. Alzheimer's disease: genes, proteins, and therapy. Physiol Rev. 2001; 81:741-766
2. Hussain I, Powell D, Howlett D R at al. Identification of a novel aspartic protease (Asp 2) as beta-secretase. Mol Cell Neurosci. 1999; 14:419-427
3. Sinha S, Anderson J P, Barbour R et al. Purification and cloning of amyloid precursor protein beta-secretase from human brain. Nature. 1999; 402:537-540
4. Vassar R, Bennett B D, Babu-Khan S et al. Beta-secretase cleavage of Alzheimer's amyloid precursor protein by the transmembrane aspartic protease BACE. Science (New York, N.Y. 1999; 286:735-741
5, Yen R, Bienkowski M J, Shuck M E et al. Membrane-anchored aspartyl protease with Alzheimer's disease beta-secretase activity. Nature. 1999; 402:533-537
6. Vassar R. Beta-secretase (BACE) as a drug target for Alzheimer's disease. Advanced drug delivery reviews. 2002; 54:1589-1602
7. Buxbaum J D, Liu K N, Luo Y et al. Evidence that tumor necrosis factor alpha converting enzyme is involved in regulated alpha-secretase cleavage of the Alzheimer amyloid protein precursor. The Journal of biological chemistry. 1998; 273:27765-27767
8. Koike H, Tomioka S, Sorimachi H et al. Membrane-anchored metalloprotease MDC9 has an alpha-secretase activity responsible for processing the amyloid precursor protein. The Biochemical journal. 1999; 343 Pt 2:371-375
9. Lammich S, Kojro E, Postina R at al. Constitutive and regulated alpha-secretase cleavage of Alzheimer's amyloid precursor protein by a disintegrin metalloprotease. Proceedings of the National Academy of Sciences of the United States of America. 1999; 96:3922-3927
10. Furukawa K, Sopher B L, Rydel R E at al. Increased activity-regulating and neuroprotective efficacy of alpha-secretase-derived secreted amyloid precursor protein conferred by a C-terminal heparin-binding domain. Journal of neurochemistry. 1996; 67:1882-1896
11. Shen J, Bronson R T, Chen D F et al. Skeletal and CNS defects in Presenilin-1-deficient mice. Cell. 1997; 89:629-639
12. Wong P C, Zheng H, Chen H at al. Presenilin 1 is required for Notch1 and DII1 expression in the paraxial mesoderm. Nature. 1997; 387:288-292
13. Luo Y, Bolon B, Kahn S et al. Mice deficient in BACE1, the Alzheimer's beta-secretase, have normal phenotype and abolished beta-amyloid generation. Nat. Neurosci. 2001; 4:231-232
14. Roberds S L, Anderson J, Basi G et al. BACE knockout mice are healthy despite lacking the primary beta-secretase activity in brain: implications for Alzheimer's disease therapeutics. Hum Mol Genet. 2001; 10:1317-1324
15. Harrison S M, Harper A J, Hawkins J at al. BACE1 (beta-secretase) transgenic and knockout mice: identification of neurochemical deficits and behavioral changes. Mol Cell Neurosci. 2003; 24:646-655
16. Willem M, Garrett A N Novak B et al. Control of peripheral nerve myelination by the beta-secretase BACE1. Science (New York, N.Y. 2006; 314:664-666
17. Sauder J M, Arthur J W, Dunbrack R L, Jr. Modeling of substrate specificity of the Alzheimer's disease amyloid precursor protein beta-secretase. Journal of molecular biology. 2000; 300:241-248
18. Hong L, Koelsch G, Lin X et al. Structure of the protease domain of memapsin 2 (beta-secretase) complexed with inhibitor. Science (New York, N.Y. 2000; 290:150-153
19. Hussain I, Hawkins J, Harrison D et al. Oral administration of a potent and selective non-peptidic BACE-1 inhibitor decreases beta-cleavage of amyloid precursor protein and amyloid-beta production in vivo. Journal of neurochemistry. 2006.

The scope of the present invention is not limited by the description, examples, and suggested uses herein and modifications can be made without departing from the spirit of the invention. Thus, it is intended that the present invention cover modifications and variations of this invention provided that they come within the scope of the appended claims and their equivalents.

1. A method for diagnosing a neurodegenerative disorder or a predisposition to develop a neurodegenerative disorder in a subject by detecting the presence of sAPPβ, a secreted β-secretase (BACE1) cleavage fragment of the beta-amyloid precursor protein (APP), in a biological sample from the subject, comprising:
(a) contacting the biological sample with an antibody that selectively binds to a BACE1 cleavage site on sAPPβ; and
(b) detecting the presence of the antibody;
wherein the presence of the antibody detected in step b) is diagnostic of a neurodegenerative disorder or a predisposition to develop a neurodegenerative disorder.

2. The method of claim 1 wherein the neurodegenerative disorder is selected from the group consisting of Alzheimer's Disease, early onset familial Alzheimer's Disease, amyotrophic lateral sclerosis (Lou Gehrig's Disease), Binswanger's Disease, corticobasal degeneration (CBD)1 dementia lacking distinctive histopathology (DLDH), frontotemporal dementia (FTD), Huntington's chorea, multiple sclerosis, myasthenia gravis, Parkinson's disease, and progressive supranuclear palsy (PSP).
